(19) United States
(12) Patent Application Publication
(10) Pub. No.: US 2022/0241487 A1
(43) Pub. Date: Aug. 4, 2022

(54) PROGRESSING ASPIRATION PUMP IN A SURGICAL SYSTEM

(71) Applicant: Johnson & Johnson Surgical Vision, Inc., Santa Ana, CA (US)

(72) Inventor: Matthew A. Sussman, Laguna Niguel, CA (US)

(21) Appl. No.: 17/597,022

(22) PCT Filed: Jun. 29, 2020
(86) PCT No.: PCT/IB2020/056123

§ 371 Date: Dec. 22, 2021

Related U.S. Application Data
(60) Provisional application No. 62/869,519, filed on Jul. 1, 2019.

(51) Int. Cl. A61M 1/00; A61F 9/007

(52) U.S. Cl. CPC A61M 1/774; A61M 1/72; A61F 9/00745

(57) ABSTRACT

An apparatus, system and method for providing a surgical handpiece. The apparatus, system and method may include: a proximal segment and a distal segment. The distal segment may include: an emulsifying tip; an irrigation output in fluidic communication with the irrigation input, and capable of supplying irrigating fluid to a surgical site; and a progressing cavity pump aspirator in fluidic communication with an aspiration port and capable of aspirating the emulsified material to the material collection point through the aspiration connection. The pump may include: a stator; and a rotor rotationally associated with the stator to effectuate pumping, via the stator cavities, of the emulsified material from the aspiration port in the distal segment through the proximal section.

### CROSS REFERENCE TO RELATED APPLICATIONS

This application claims priority to U.S. Provisional Patent Application No. 62/869,519, filed on Jul. 1, 2019, the entire contents of which are hereby incorporated by reference.

### BACKGROUND

### Field of the Disclosure

The present disclosure relates to medical devices and systems, and, more specifically, to an apparatus, system and method of providing a progressing aspiration pump in a surgical system.

### Description of the Background

Phacoemulsification is a medically recognized technique utilized for crystalline lens removal. Phacoemulsification includes making a corneal and/or scleral incision, and the insertion of a phacoemulsification handpiece, which is typically comprised of a needle that is ultrasonically driven in order to emulsify, i.e., to liquefy, the natural crystalline lens and/or an unhealthy aspect, such as a cataract, associated therewith.

The phacoemulsification handpiece is generally coupled to an irrigation source and an aspiration pump. As referenced, the handpiece includes a distal tip for insertion within the anterior chamber of the patient's eye and which uses ultrasonic energy to emulsify the crystalline lens. The handpiece further includes one or more irrigation ports proximal to the distal tip, which is coupled to the irrigation source via an irrigation line, and an aspiration port at the distal tip, which is coupled to the aspiration pump via an aspiration line. Fluid from the irrigation source, which may be, by way of non-limiting example, an elevated bottle of saline solution, or a pressurized or gas-forced infusion, is irrigated into the eye via the irrigation line and the irrigation port(s), and the irrigation fluid and emulsified crystalline lens material and fluid are aspirated from the eye by the aspiration pump via the aspiration port and the aspiration line.

There are a variety of other, similar medical techniques that are applied to the unhealthy eye, and each such technique also typically includes irrigating the eye and aspirating at least the irrigation fluid. Such procedures may or may not include the destruction, alteration or removal of features of the natural eye.

Aspiration is generally achieved with one or more of a variety of different aspiration pumps known in the art. Two common types of aspiration pumps are: volumetric flow, or positive displacement, pumps (such as peristaltic or scroll pumps); and vacuum-based, or non-positive displacement, pumps (such as venturi, diaphragm, or rotary-vane pumps).

A vacuum-based aspiration pump indirectly controls fluid flowby controlling the vacuum within the fluidic circuit. For example, a venturi pump creates a lower pressure in a cassette reservoir, which causes the fluid to flow from the eye into the aspiration line and through to the cassette reservoir. Thus, instead of pushing fluid through the aspiration line like a volumetric flow pump, the fluid is essentially pulled by a vacuum through the line. The rate of fluid flow generated by a vacuum-based pump is generally higher than the rate of fluid flow generated by a volumetric flow-based pump, but current systems and methods for controlling the rate of flow for the vacuum-based pump necessitate manual adjustment of the operative vacuum level.

Moreover, during phacoemulsification in particular, it is possible for the aspirating phacoemulsification handpiece to become occluded by lens particles that blocks the distal tip of aspirating handpiece. For volumetric flow pumps, this blockage can result in the creation or increase in vacuum. For a vacuum-based pump, this blockage can result in a volumetric fluid flow drop off near the aspiration port. In each such case, once the occlusion is cleared, the resulting rush of fluid from the anterior chamber into the aspiration line can outpace the volumetric flow of new fluid into the eye from the irrigation source, which may lead to severe eye trauma.

Therefore, the need exists for an apparatus, system, and method for providing more controllable vacuum in a surgical system, such as to avoid severe eye trauma.

### SUMMARY

The disclosed apparatus, system and method is and may include at least a surgical handpiece that includes: a proximal segment having an aspiration connection in fluidic communication with a material collection point; an irrigation input; and a power input. Also included in the surgical handpiece is a distal segment, which includes: an emulsifying tip driven by power from the power input capable of emulsifying material; an irrigation output in fluidic communication with the irrigation input, and capable of supplying irrigating fluid to a surgical site; and a progressing cavity pump aspirator in fluidic communication with an aspiration port and capable of aspirating the emulsified material (and irrigation fluid) to the material collection point through the aspiration connection. The pump may include: a stator having a plurality of at least substantially circumferential cavities; and a rotor associated with the stator to effectuate pumping via the at least substantially circumferential cavities of the emulsified material from the aspiration port to the aspiration connection.

Thus, the disclosed embodiments provide an apparatus, system, and method for providing more controllable vacuum in a surgical system, such as to avoid severe eye trauma.

### BRIEF DESCRIPTION OF THE FIGURES

Referring now to the figures incorporated herein, shown are non-limiting embodiments of the present disclosure, wherein like numerals may, but do not necessarily, represent like elements, and wherein:

FIG. 1 is an illustration of aspects of the embodiments;

FIG. 2 is an illustration of aspects of the embodiments;

FIG. 3 is an illustration of aspects of the embodiments;

FIG. 4 is an illustration of aspects of the embodiments;

FIG. 5 is an illustration of aspects of the embodiments;

FIG. 6 is an illustration of aspects of the embodiments;

FIGS. 7A and 7B are illustrations of aspects of the embodiments;

FIG. 8 is an illustration of aspects of the embodiments;

FIG. 9 is an illustration of aspects of the embodiments;

FIG. 10 is an illustration of aspects of the embodiments; and

FIG. 11 is an illustration of aspects of the embodiments.

### DETAILED DESCRIPTION

The figures and descriptions provided herein may have been simplified to illustrate aspects that are relevant for a clear understanding of the herein described apparatuses, systems, and methods, while eliminating, for the purpose of clarity, other aspects that may be found in typical similar devices, systems, and methods. Those of ordinary skill may thus recognize that other elements and/or operations may be desirable and/or necessary to implement the devices, systems, and methods described herein. But because such elements and operations are known in the art, and because they do not facilitate a better understanding of the present disclosure, for the sake of brevity a discussion of such elements and operations may not be provided herein. However, the present disclosure is deemed to nevertheless include all such elements, variations, and modifications to the described aspects that would be known to those of ordinary skill in the art.

Exemplary embodiments are provided throughout so that this disclosure is sufficiently thorough and fully conveys the scope of the disclosed embodiments to those who are skilled in the art. Numerous specific details are set forth, such as examples of specific components, devices, and methods, to provide a thorough understanding of embodiments of the present disclosure. Nevertheless, it will be apparent to those skilled in the art that certain specific disclosed details need not be employed, and that exemplary embodiments may be embodied in different forms. As such, the exemplary embodiments should not be construed to limit the scope of the disclosure. As referenced above, in some exemplary embodiments, well-known processes, well-known device structures, and well-known technologies may not be described in detail.

The terminology used herein is for the purpose of describing particular exemplary embodiments only and is not intended to be limiting. For example, as used herein, the singular forms “a”, “an” and “the” may be intended to include the plural forms as well, unless the context clearly indicates otherwise. The terms “comprises,” “comprising” “including,” and “having,” are inclusive and therefore specify the presence of stated features, integers, steps, operations, elements, and/or components, but do not preclude the presence or addition of one or more other features, integers, steps, operations, elements, components, and/or groups thereof. The steps, processes, and operations described herein are not to be construed as necessarily requiring their respective performance in the particular order discussed or illustrated, unless specifically identified as a preferred or required order of performance. It is also to be understood that additional or alternative steps may be employed, in place of or in conjunction with the disclosed aspects.

When an element or layer is referred to as being “on”, “engaged to”, “connected to” or “coupled to” another element or layer, it may be directly on, engaged, connected or coupled to the other element or layer, or intervening elements or layers may be present, unless clearly indicated otherwise. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). Further, as used herein the term “and/or” includes any and all combinations of one or more of the associated listed items.

Yet further, although the terms first, second, third, etc. may be used herein to describe various elements, components, regions, layers and/or sections, these elements, components, regions, layers and/or sections should not be limited by these terms. These terms may be only used to distinguish one element, component, region, layer or section from another element, component, region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the exemplary embodiments.

Certain types of ocular dysfunction, such as cataracts, are commonly treated with surgical procedures, such as to remove the natural lens from the eye and replace it with a clear artificial lens. More particularly and by way of example, phacoemulsification refers to a surgery, often employed when a patient suffers from cataracts, in which the eye's natural lens is emulsified by applying ultrasonic energy to the lens using a handpiece. Once the lens is emulsified, it is aspirated from the eye by applying a vacuum to the emulsified lens material. During the procedure, irrigation is performed, and aspirated material replaced, using an irrigating fluid, such as a balanced salt solution, thereby maintaining pressure in the interior of the eye. The emulsified and aspirated lens is then typically replaced with a clear artificial intraocular lens (IOL).

To perform the afore-discussed and similar procedures, a surgeon often utilizes a computer-controlled system of specialized equipment called a phacoemulsification system console to control and execute the ultrasonic emulsification and aspiration of the natural lens of the eye prior to inserting the IOL. During the procedure, information such as the amount of vacuum applied to aspirate, the flow rate, a microscopic view of the operating field, and the like, may be displayed on and controllable from a user interface of the phacoemulsification system console, or on a separate screen, computer, or other viewing device, and may be monitored and verbally reported by support staff during the procedure. At least some of this data is commonly used to inform and improve ongoing and subsequent procedures.

In phacoemulsification, the ultrasonic vibration of the handpiece tip is generally paired with the irrigating fluid flow and the aspiration in order to safely and effectively perform the surgery, as discussed throughout. In a typical cataract surgery, by way of example, the vacuum pulls the cataract up to the vibrating tip and holds it in place to be broken down into pieces small enough to be aspirated via the handpiece out of the eye. However, a common issue referred to as post occlusion surge may lead to severe trauma during performance of the surgery.

Post occlusion surge occurs when the tip of a needle of a handpiece is temporarily blocked or partially blocked, such as by the emulsified material, and the vacuum consequently builds between the handpiece and the vacuum pump. Exacerbating this issue, most types of vacuum pumps operate with pulsation, and/or otherwise provide a varying flow rate. The foregoing creates a lack of consistency in operation during phacoemulsification and similar procedures, and thus increases the surgical difficulty for the surgeons while decreasing the safety of patients, particularly upon occurrence of an occlusion.

Disclosed is a progressing cavity pumping phacoemulsification handpiece, which may use ultrasound to break up cataracts or malformities of the eye, as is known in the present art. However, the disclosed embodiments include, integrated into the handpiece, a progressing cavity pump to aspirate the emulsified material. This progressing cavity pump does not pulse, and is thus capable of maintaining flow rates to a higher degree of accuracy than is known in the pertinent arts. The disclosed handpiece may be substantially self-sealing, and may substantially or completely eliminate post occlusion surge. Of course, one of ordinary skill in the pertinent arts will appreciate that the disclosed embodiments may be used in other contexts, such as in a dosing pump, by way of non-limiting example.

The disclosed handpiece embodiments may be a multi-piece, such as a two-piece, design. Ones of the multiple pieces may be 3D printed, such as using titanium print material and/or extruded. The multiple sections of the housing are connected, such as using bayonet-style, spring and clip, threaded, or other connection methodologies, such as in conjunction with one or more mechanical seals or a Luer lock, by way of example. This type of construction may allow for the handpiece to be easily disassembled, such as for cleaning such as using autoclaving.

In some embodiments, the stator for the progressing pump may be integrated into a front section of the handpiece, and may also be constructed via 3D printing, such as using the same printed titanium material referenced above. The rotor may be, by way of non-limiting example, an elastomer. The rotor may be fitted onto a shaft. The shaft may comprise an Oldham coupling, k-type coupling, an elastomer coupling, or a short flexible rotary coupling, by way of example, to account for the eccentric rotation of the rotor, by way of non-limiting example. The rotor may be actuated by a flexible rotary shaft connected to a motor, such as may be located at the phacoemulsification console. Of course, a direct mechanical coupling of the motor may likewise be used.

In additional optional embodiments, a planetary gear set in the handpiece may step down the high RPM of the flexible shaft to the higher torque requirements necessary to run the rotor in, for example, a phacoemulsification embodiment. Moreover, the irrigation and aspiration discussed throughout may be fluidically connected to a rear section of the handpiece using, for example, Luer fittings to integrated Luer adapters on the handpiece.

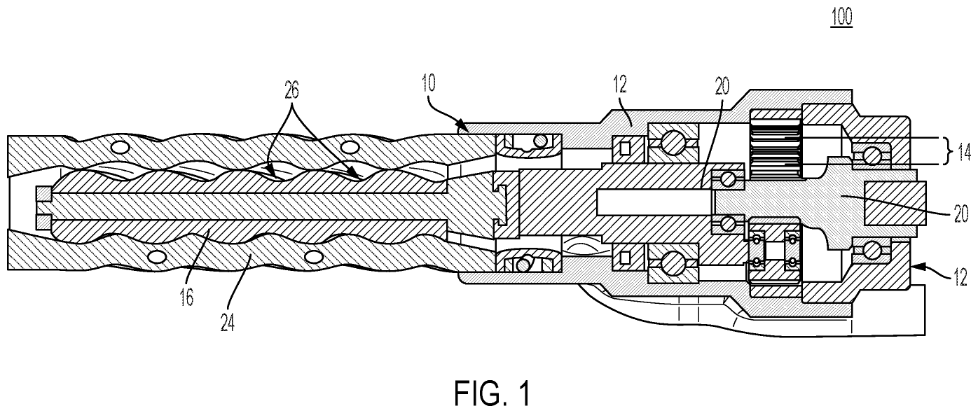

As referenced above, there are a variety of different types of pumps available in the known art. Non-positive displacement pumps include venturi-type pumps and rotary-type pumps, and positive displacement pumps include reciprocating pumps, progressing cavity pumps, and rotary piston pumps, by way of non-limiting example. In the presently described embodiments, a progressing cavity pump may be employed as discussed throughout. For example, FIG. 1 illustrates a progressing cavity pump 10 and a proximal portion 12 of a phacoemulsification hand piece 100 in a cross-sectional view.

In the illustration, a plurality of gears 14 may be provided, such as in eventual mechanical association with a rotor 16 to drive the progressing pump 10. Of note, the cavity pump rotor 16 may be of an elastomer composition, although other compositions apparent to the skilled artisan in light of the discussion herein may be employed without departing from the embodiments.

As mentioned, the rotor 16 may be driven by mechanical association with the gearing 14, such as via with a motor shaft 20. That is, the gearing 14 may mechanically associate with a shaft 20 which, upon rotation, directly or indirectly causes the progressing cavity pump rotor 16 to rotate, thus driving pumping by pump 10.

This pumping may occur in that the disclosed shaft 20 and rotor 16 may cause rotation of the rotor 16 within a progressing cavity pump stator 24 having a plurality of circumferential stator cavities 26. These cavities 26 effectuate the pumping action in conjunction with the turning of the rotor 16, as will be understood by the skilled artisan in light of the discussion herein.

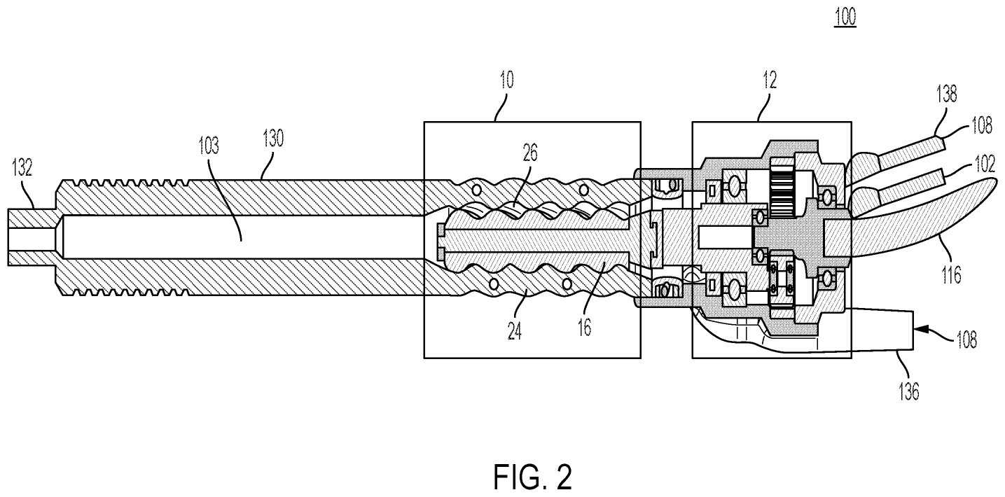

FIG. 2 illustrates a handpiece 100 comprised of the progressing cavity pump portion 10 of a handpiece 100; a proximal portion 12 of the hand piece 100 having associated therewith at least one power input 102 to drive the emulsifying ultrasonic aspect 103 of the hand piece 100 (and, in some embodiments, the progressing cavity pump 10 or the corresponding motor/gears described above and throughout); as well as aspiration output and irrigation connections 108 (noting that irrigation may employ a sleeve coupled with the distal end of the hand piece and at least partially surrounding the needle to allow irrigation fluid to exit the handpiece and enter the anterior chamber of the eye) to the proximal end 12 of the hand piece 100. It will be appreciated that, while aspects of the disclosure are provided in relation to ultrasonics, other types of lens emulsifying features may be used without departing from the disclosure. Also illustrated by way of alternative discussion, in FIG. 2 is a flexible shaft 116 to drive the progressing pump 10, as is discussed herein throughout.

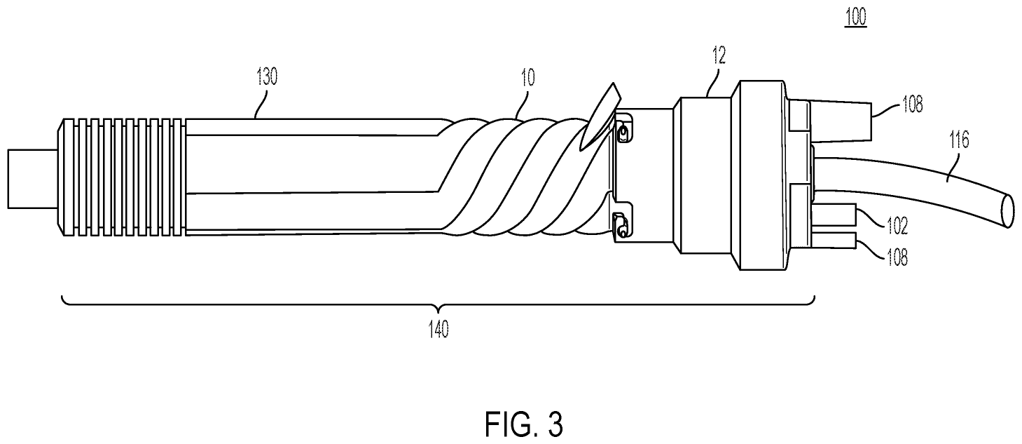

As shown, the progressing pump portion 10 of the disclosed hand piece 100 may physically associate with a distal portion 130 of the hand piece 100, which includes: the needle 132 driven by an ultrasonic transducer 103; as well as ports 136, 138 (in connections 108) to provide the aspiration and irrigation into and out of the eye as discussed herein. FIG. 3 is a profile view of an exemplary enclosure 140 provided by the combination of the outer aspects of the afore-discussed portions, namely the outer aspects of distal portion 130, progressing cavity pump portion 10, and proximal portion 12 of the hand piece 100, which are collectively illustrated in cross-section in FIG. 2.

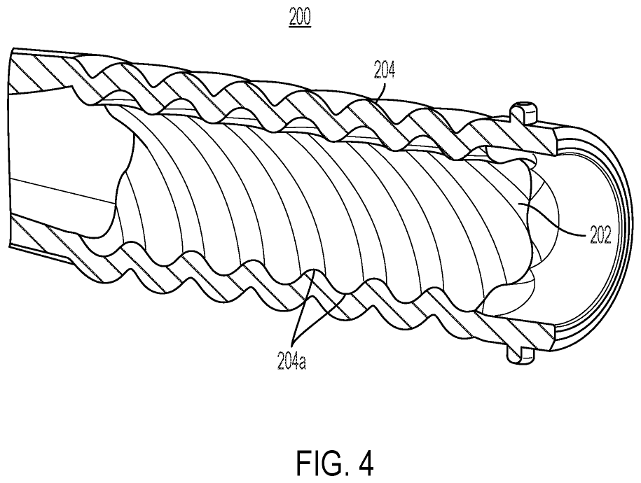

FIG. 4 illustrates a cross-section of an exemplary rotor 202 and stator 204 for a progressing cavity pump portion 200 of a handpiece for use in the embodiments. In the illustration, the rotor 202 may be of any known composition, including an elastomer, by way of non-limiting example. The rotor 202 may be a one-half, two-thirds, three-fourths, or four-fifths ratio lobe geometry, by way of non-limiting example, and the skilled artisan will appreciate that, as the number of lobes increases, the cavity size may decrease, thereby increasing the flow rate available. Pump 200 may be a single or a multi-stage pump, and the number of stages in multi-stage embodiments may vary, such as 1.5 stages in certain embodiments.

The stator 204 illustrated in FIG. 4 may also be comprised of any known material, including a printed composition, such as a printed metal, for example titanium or stainless steel. Of note, the stator 204 may include a finishing 204a on surfaces where the stator 204 comes in contact with the rotor 202, such as may improve pumping capabilities. Such a finish 204a may be imparted by any known methodology, such as through the use of stator-negative drill bits, by way of non-limiting example.

The described stator and rotor system may include various other aspects known in the art to be provided in such pumping systems. By way of example, the stator may include a through-port for bleeding as needed. Yet further, the stator-rotor system may include any one or more of pressure, RPM, pump volume, or like-sensor monitoring, by way of non-limiting example, such as may be communicatively associated with the console referenced throughout.

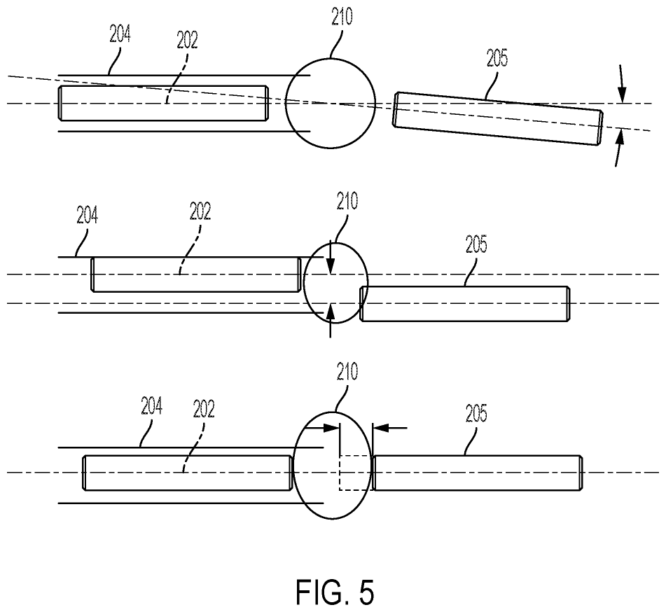

With reference now to FIGS. 3, 4, and 5 it will be noted that a shaft coupling 210 may be necessary due to non-axial rotational variations of the rotor 202 within the stator 204 due to connections to the shaft 205. The shaft coupling 210 may comprise an Oldham coupling k-type coupling, an elastomer coupling, or a short flexible rotary coupling, by way of non-limiting example. The shaft coupling 210 may provide a flex coupling, or may employ a flexible shaft, by way of non-limiting example. This non-axial rotation may be caused by any of a variety of relationships between the shaft at the coupling and the rotor, such as due to angular misalignment, radial misalignment, or axial displacement, which are also illustrated with particularity in FIG. 5.

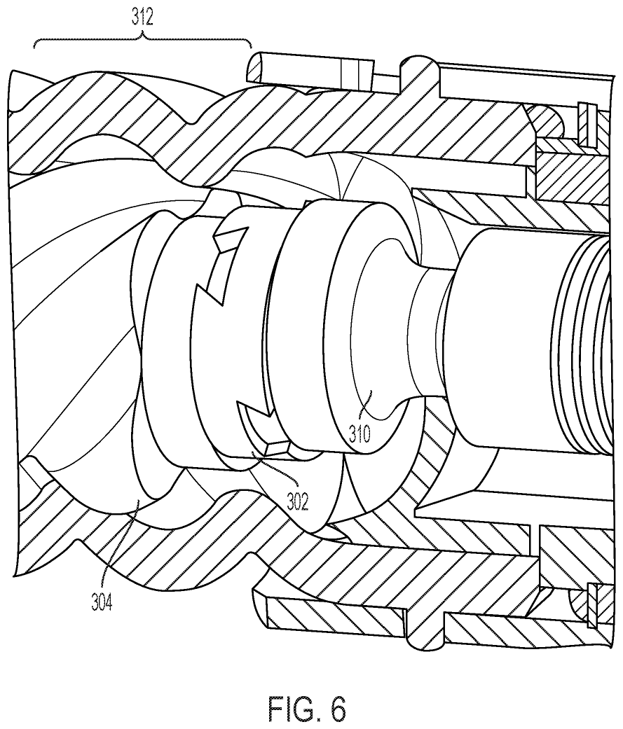

A variety of coupling types 302 may be used in various of the embodiments to associated a driveshaft 310 with a rotor 304. By way of example, dovetail coupling 302 is illustrated in FIG. 6. In the illustration, the dovetail 302 may allow for slip fitting or press fitting of the driveshaft 310 into association with the rotor 304 of the rotor and stator system 312.

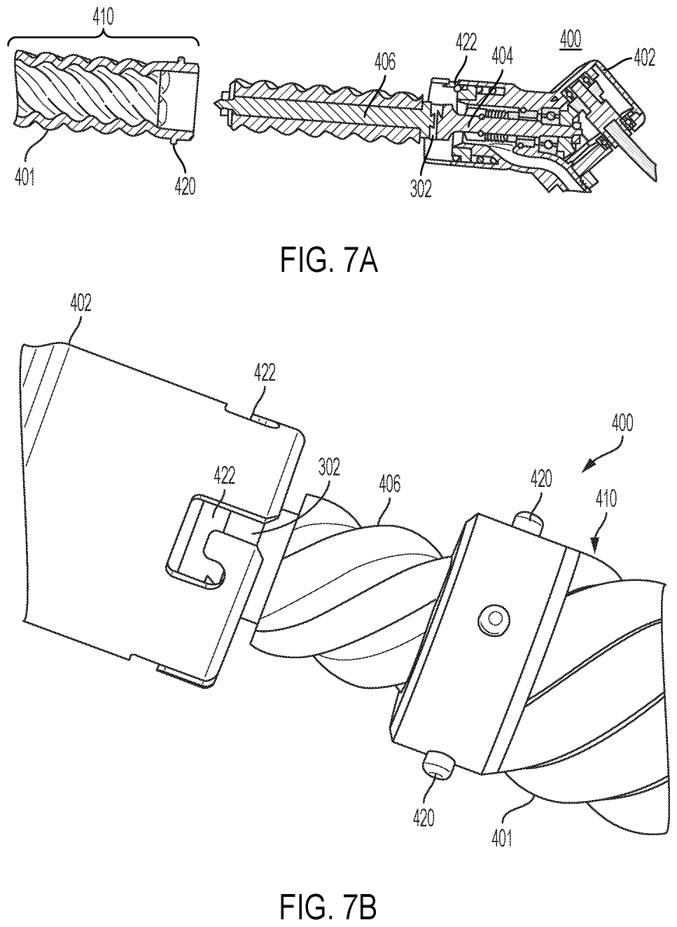

FIGS. 7A and 7B illustrate the association of one portion of the hand piece 400, comprising the proximal segment 402 and the shaft 404 and rotor 406 of the progressing pump, with a second segment 410 of the hand piece 400, including stator 401. As illustrated with particularity in FIG. 7B, the association of these two segments 402, 410 of the hand piece may include a plurality of spring clips 420 about the circumference of a first segment of the hand piece at the uppermost portion of the rotor 406, and mating spring slots 422 on a second segment of the hand piece 400.

Needless to say, in the immediately foregoing embodiment, the skilled artisan will appreciate that mating feature of the two segments 402, 410 of the hand piece 400 must be designed so as to allow for sufficient downward travel of the spring clips 420 into the “C”-shaped portion of the spring slots 422, which may require a particular spring strength as between second segment 410 and proximal segment 402 so as to upwardly lock spring clips 420 into the end of the C clip portion of the spring slots 422 to lock the hand piece segments 402, 410 together. Of course, it will be appreciated that the coupling 420, 422 between segments 402, 410 may comprise other known coupler types, such as a bayonet style coupling having aspects on each segment 402, 410, threaded couplers, tab and clip couplers, and so on.

It will further be appreciated that various seals or sealing materials, as well as various rotation-enabling materials, such as washers, may be associated with the segment coupler (e.g. spring clips 420 and spring slots 422 discussed herein), without departing from the disclosure. These seals, sealing materials and aforementioned coupling materials, may be of any known composition, including comprising 3D printed and/or extruded elements, by way of example. Of note, either the spring clips 420, spring slots 422, or both of the aforementioned couplers may be 3D printed, such as being formed of printed titanium or stainless steel or other similar material, by way of non-limiting example.

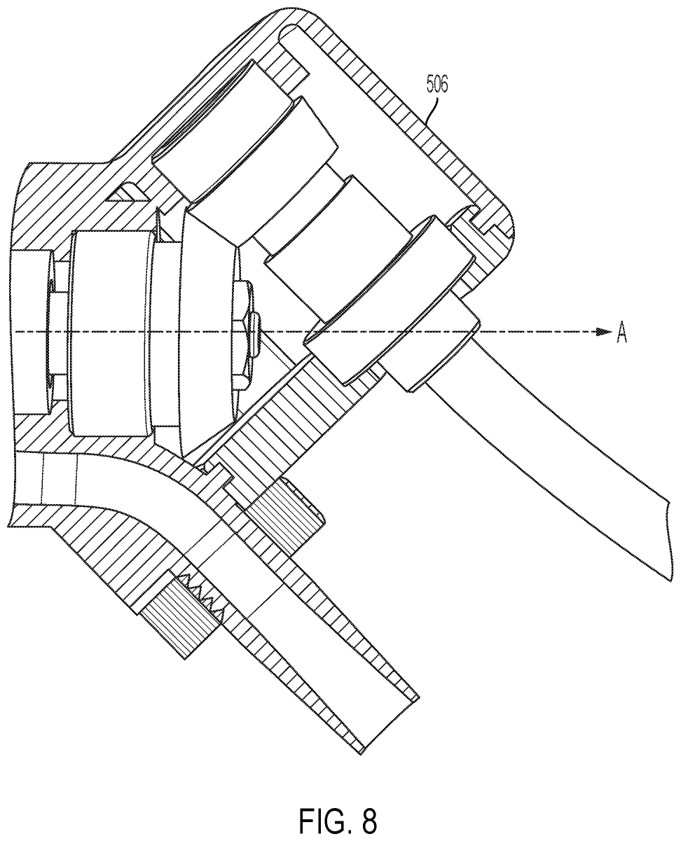

As referenced above, the rotor 406 may be mechanically communicative with a geared driveshaft 404, such as may be associated with a gearbox and/or a drive motor. FIG. 8 illustrates an angled one of such a drivebox 506. By way of non-limiting example, the angle of the drivebox 506 may be 45 degrees from axis A.

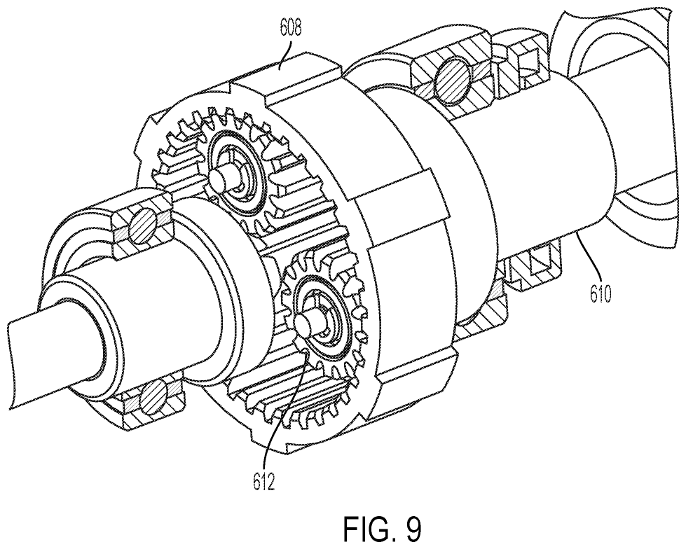

FIG. 9 illustrates an additional embodiment having a linear gearbox 608 and driveshaft 610. The linear gearbox 608 may employ, by way of non-limiting example, planetary gearing 612 in driving driveshaft 610.

Also associated with any or all of the foregoing embodiments may be a detachable and/or flexible shaft suitable to impart rotation to the disclosed gears. Such a detachable and/or flexible shaft may impart the advantages discussed throughout. Moreover, it should be noted that one or more of the gears disclosed herein, or other aspects, may be 3D printed, such as using similar processes to those employed to print the aspects discussed above.

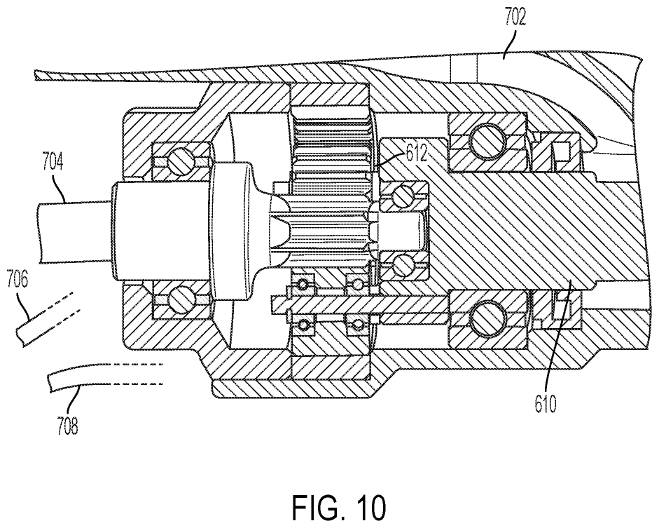

FIG. 10 illustrates an aspiration connector 708 to receive aspirated material pumped by the disclosed progressing cavity pump, and a linear, flexible driveshaft input 704 in accordance with the foregoing embodiments. Also of note, in conjunction with FIG. 10 and the other embodiments disclosed herein, it will be appreciated that power connection 706 and irrigation line 702 are also associated with the disclosed hand piece to the extent the hand piece is deployed for the purposes of phacoemulsification. In such instances, the power connection and irrigation line may be connected with the proximal portion of the hand piece discussed herein throughout; may be associated with the segment connection discussed herein above; or may be connected at the distal portion of the hand piece. In each such embodiment, cabling and tubing may be managed using known methodologies, such as swivel joints, mechanical cable managers, magnetics, flexible or rigid cabling and tubing and the like.

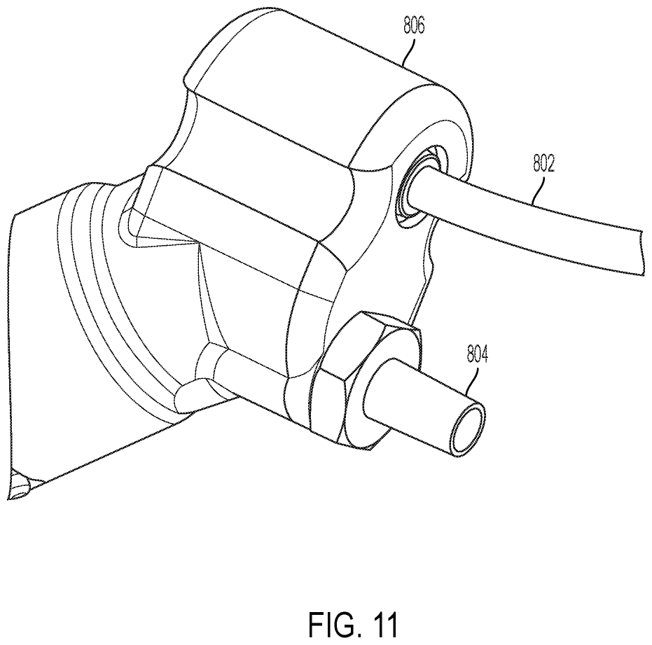

FIG. 11 additionally provides an illustration of a flexible driveshaft input 802 and an aspiration connector or port 804. In the illustration of FIG. 11, an angled gearbox 806 such as that discussed above in FIG. 8 is employed. Of note, a linear gearbox, such as that discussed with respect to FIGS. 9 and 10, may also be used. In either case, the use of a flexible driveshaft input 802 may allow for implementation of the minimum torqueing RPM necessary in the performance of particular embodiments.

In alternative embodiments to those discussed above, magnetic drive of the drive system for the rotor may eliminate the need for a mechanical seal, as no mechanical interaction between the drive in the rotor system is needed, and may further eliminate the need for a flexible shaft. Such an embodiment may include the use of the permanent magnetic A/C motor in the drive system, and may allow for rotation of the stator rather than rotation of the rotor.

In the foregoing detailed description, it may be that various features are grouped together in individual embodiments for the purpose of brevity in the disclosure. This method of disclosure is not to be interpreted as reflecting an intention that any subsequently claimed embodiments require more features than are expressly recited.

Further, the descriptions of the disclosure are provided to enable any person skilled in the art to make or use the disclosed embodiments. Various modifications to the disclosure will be readily apparent to those skilled in the art, and the generic principles defined herein may be applied to other variations without departing from the spirit or scope of the disclosure. Thus, the disclosure is not intended to be limited to the examples and designs described herein, but rather is to be accorded the widest scope consistent with the principles and novel features disclosed herein.

What is claimed is:

1. A surgical handpiece, comprising:
a proximal segment, comprising:
an aspiration connection in fluidic communication with a material collection point;
an irrigation input; and
a power input;
a distal segment, comprising:
an emulsifying tip driven by power from the power input capable of emulsifying material;
an irrigation output in fluidic communication with the irrigation input, and capable of supplying irrigating fluid to a surgical site; and
a progressing cavity pump aspirator in fluidic communication with an aspiration port and capable of aspirating the emulsified material through the aspiration connection to the material collection point, wherein the progressing cavity pump aspirator comprises:
a stator having a plurality of at least substantially circumferential cavities; and
a rotor associated with the stator and configured to effectuate pumping via the at least substantially circumferential cavities of the emulsified material from the aspiration port to the aspiration connection.

2. The handpiece of claim 1, wherein the rotor is configured to rotate and the stator is stationary.

3. The handpiece of claim 1, wherein the stator is configured to rotate and the rotor is stationary.

4. The handpiece of claim 1, wherein the aspiration connection and the irrigation input comprise Luer fittings.

5. The handpiece of claim 1, wherein the proximal end further comprises a plurality of gears coupled with the rotor.

6. The handpiece of claim 5, wherein the proximal end further comprises a mechanical connection to a motor, wherein the motor is configured to drive the plurality of gears.

7. The handpiece of claim 1, wherein the proximal end further comprises a shaft in mechanical association with the rotor and configured to drive rotation of the rotor.

8. The handpiece of claim 7, wherein the shaft is flexible.

9. The handpiece of claim 7, wherein the shaft is flexibly connected to the rotor.

10. The handpiece of claim 1, wherein the rotor comprises an elastomer.

11. The handpiece of claim 1, further comprising a coupler capable of coupling the proximal segment to the distal segment.

12. The handpiece of claim 11, wherein the coupler comprises at least two spring clips on the proximal segment and receiving spring slots on the distal segment.

13. The handpiece of claim 12, wherein the coupler comprises one selected from the group of mated threadings, paired bayonets and receivers, and paired tabs and slots.

14. The handpiece of claim 1, wherein the rotor comprises one of a one-half, two-thirds, three-fourths, and four-fifths ratio lobe geometry.

15. The handpiece of claim 1, wherein the stator comprises one of a printed titanium and a printed steel.

16. The handpiece of claim 1, wherein the stator comprises a finishing capable of receiving the rotor.
